(19) United States
(12) Patent Application Publication
(10) Pub. No.: US 2025/0082281 A1
(43) Pub. Date: Mar. 13, 2025

(54) TECHNIQUES FOR DETERMINING OPTICAL PERFORMANCE OF A WEARABLE DEVICE

(71) Applicant: Oura Health Oy, Oulu (FI)

(72) Inventor: Jaakko Vartiainen, Oulu (FI)

(21) Appl. No.: 18/462,731

(22) Filed: Sep. 7, 2023

(51) Int. Cl. A61B 5/00

(52) U.S. Cl. CPC A61B 5/7282; A61B 5/0064; A61B 5/6826

(57) ABSTRACT

Methods, systems, and devices for determining optical performance of a wearable device are described. The method may include a wearable device receiving, from a charging unit coupled with the wearable device, an electrical input for charging a battery of the wearable device and activating, while the wearable device is coupled with the charging unit, optical components of the wearable device. Further, the method may include generating a first signal based on light received at an optical component of the optical components of the wearable device and measuring a performance metric associated with the wearable device based on the first signal. Further, the method may include transmitting a second signal indicating the performance metric to a user device associated with the wearable device.

### FIELD OF TECHNOLOGY

The following relates to wearable devices and data processing, including techniques for determining optical performance of a wearable device.

### BACKGROUND

Some wearable devices may be configured to collect data, such as physiological data, from a user of the wearable device. For example, the wearable device may include one or more optical components to collect physiological data from the user. In some examples, the one or more optical components may degrade in performance over time or due to manufacturing errors which may affect the accuracy of the physiological data being provided to the user of the wearable device.

### BRIEF DESCRIPTION OF THE DRAWINGS

FIG. 1 illustrates an example of a system that supports techniques for determining optical performance of a wearable device in accordance with aspects of the present disclosure.

FIG. 2 illustrates an example of a system that supports techniques for determining optical performance of a wearable device in accordance with aspects of the present disclosure.

FIG. 3 shows an example of a system that supports techniques for determining optical performance of a wearable device in accordance with aspects of the present disclosure.

FIG. 4 shows a block diagram of an apparatus that supports techniques for determining optical performance of a wearable device in accordance with aspects of the present disclosure.

FIG. 5 shows a block diagram of a wearable device manager that supports techniques for determining optical performance of a wearable device in accordance with aspects of the present disclosure.

FIG. 6 shows a diagram of a system including a device that supports techniques for determining optical performance of a wearable device in accordance with aspects of the present disclosure.

FIG. 7 shows a block diagram of an apparatus that supports techniques for determining optical performance of a wearable device in accordance with aspects of the present disclosure.

FIG. 8 shows a block diagram of a wearable application that supports techniques for determining optical performance of a wearable device in accordance with aspects of the present disclosure.

FIG. 9 shows a diagram of a system including a device that supports techniques for determining optical performance of a wearable device in accordance with aspects of the present disclosure.

FIGS. 10 through 14 show flow charts illustrating methods that support techniques for determining optical performance of a wearable device in accordance with aspects of the present disclosure.

### DETAILED DESCRIPTION

In some examples, a wearable device may utilize optical components (e.g., light-emitting components or photodetectors) to collect physiological data for a user of the wearable device. As such, the user may expect the wearable device to provide accurate physiological data. However, multiple factors may decrease the performance and accuracy of the optical components of the wearable device. For example, over time the optical components may become weaker or degrade. As another example, during manufacturing, defects (e.g., bubbles or craters) in a material (e.g., epoxy) located in or around the optical component may obstruct or alter the optical components. Further, debris (e.g., dust or dirt) may cover the optical components resulting in less light being received or transmitted by the optical components. Currently, there is no method to track or identify performance changes of the optical components of the wearable device which may promote inaccurate physiological data being provided to the user.

As described herein, the wearable device may measure the performance of the optical components while connected to a charging unit and utilize these measurements to adjust or calibrate algorithms used for determining physiological data. Using at least one light-emitting component, the wearable device may transmit light and using at least one photodetector, the wearable device may receive the light. Upon receiving the light, the wearable device may determine a signal level of the received light and a noise level of the received light and transmit a message to a user device associated with the wearable device indicating the signal level and noise level of the received light. Based on the message, the user device may determine whether updates to the optical component pair are needed. For example, the user device may compare the signal level and/or noise level of the received light to a threshold (e.g., a baseline threshold). If the signal level and/or the noise level is below the threshold, the user device may determine the optical performance of the optical component pair is poor and may instruct the wearable device to adjust one or more measurement parameters associated with the optical component pair (e.g., a transmit power of the light-emitting component, among other measurement parameters).

Aspects of the disclosure are initially described in the context of systems supporting physiological data collection from users via wearable devices. Aspects of the disclosure are further illustrated by and described with reference to apparatus diagrams, system diagrams, and flowcharts that relate to techniques for determining optical performance of a wearable device.

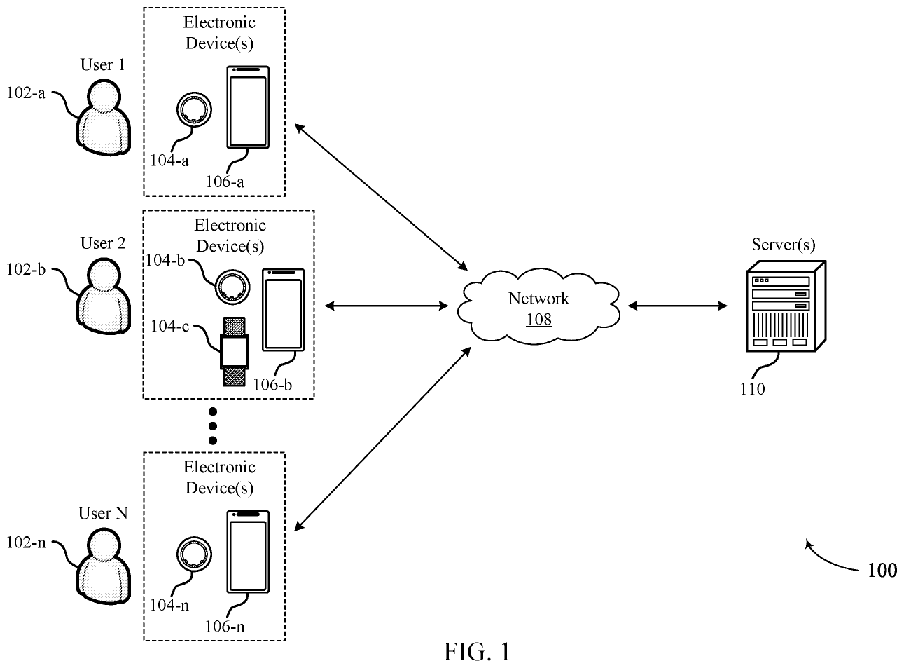

FIG. 1 illustrates an example of a system 100 that supports techniques for determining optical performance of a wearable device in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, blood oxygen saturation (SpO2), blood sugar levels (e.g., glucose metrics), and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols.

In some implementations, the rings 104 (e.g., wearable devices 104) of the system 100 may be configured to collect physiological data from the respective users 102 based on arterial blood flow within the user's finger. In particular, a ring 104 may utilize one or more light-emitting components, such as LEDs (e.g., red LEDs, green LEDs) that emit light on the palm-side of a user's finger to collect physiological data based on arterial blood flow within the user's finger. In general, the terms light-emitting components, light-emitting elements, and like terms, may include, but are not limited to, LEDs, micro LEDs, mini LEDs, laser diodes (LDs) (e.g., vertical cavity surface-emitting lasers (VCSELs), and the like.

In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time that a user 102 is asleep, and classify periods of time that the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time that the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g., in a hypothetical culture with 12 day “weeks,” 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

As described herein, the wearable device 104 may perform measurements on optical components such as photodiode and LEDs while connected to a charging unit. In some examples, the wearable device 104 may include a rechargeable battery that is operable to provide power to one or more components of the wearable device 104. After continued use of the wearable device 104, the rechargeable battery may lose charge and as a result, the wearable device 104 may be connected to a charging unit which may recharge the rechargeable battery. While connected to the charging unit, the wearable device 104 may activate one or more optical components of the wearable device 104. For example, the wearable device 104 may activate an LED and a photodiode. In some examples, the wearable device 104 may be an example of a ring and the LED and photodiode may be situated along an inner surface (or inner housing 205-a) of the ring.

Activating the LED and the photodiode may include transmitting light via the LED and receiving the transmitted light via the photodiode. In some examples, the light may be transmitted into an open space between the charging unit and the wearable device 104. The wearable device 104 may analyze the light received at the photodiode. For example, the wearable device 104 may determine an average signal strength of the received light over time or an average change in signal strength of the received light over time (or average noise). In some examples, the average signal strength or the average change in signal strength may be known as a performance metric for the LED and photodiode pair.

Further, the wearable device 104 may transmit the performance metric to the user device 102 associated with the user 102 and the user device 106 may determine a performance of the LED and photodiode pair based on the performance metric. For example, the user device 102 may determine that the LED and photodiode are exhibiting poor performance based on the performance metrics being less than a threshold. The threshold may be a baseline threshold that is based on a performance metric determined during manufacture testing or a previously determined performance metric. In some examples, the baseline threshold may be stored at one or both of the user device 106 or the server 110.

If the user device 106 determines the performance of the LED and photodiode pair is poor, the user device 106 may transmit instructions to the wearable device 104 regarding the LED and photodiode pair. As one example, the user device 106 may instruct the wearable device 104 to adjust a transmit power of the LED, a burn time of the LED, or an algorithm for acquiring physiological data for the user 102. After receiving the instruction, the wearable device 104 may perform physiological measurements for the user 102 according to the adjustment.

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

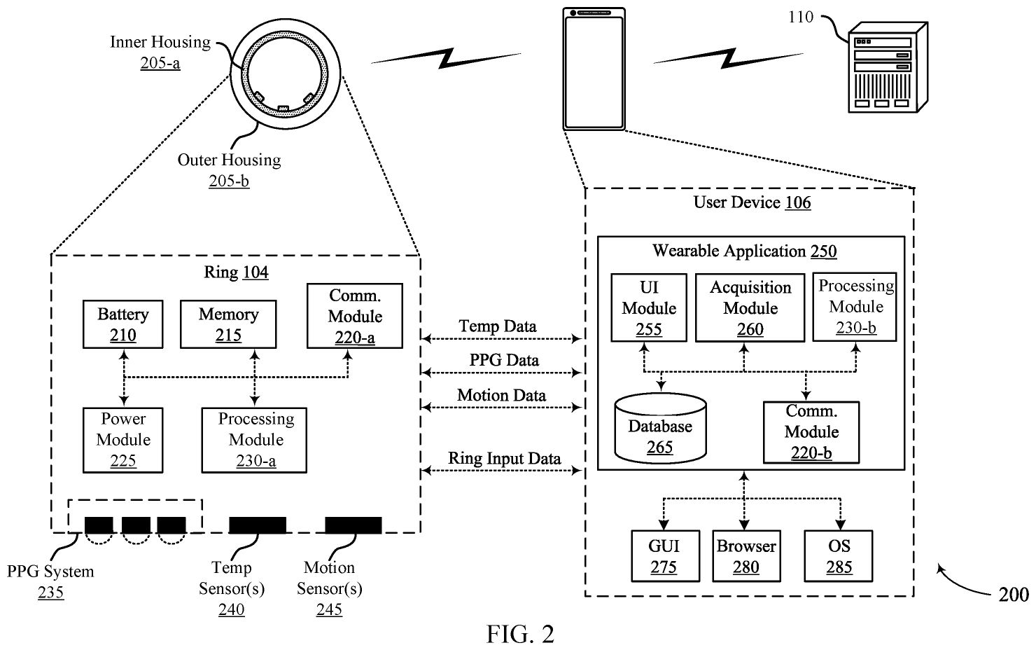

FIG. 2 illustrates an example of a system 200 that supports techniques for determining optical performance of a wearable device in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels (SpO2), blood sugar levels (e.g., glucose metrics), and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, photoplethysmogram (PPG) data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205 that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using adhesives, wraps, clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or that supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during charging, and under voltage during discharge. The power module 225 may also include electro-static discharge (ESD) protection.

In some examples, while the ring 104 is interfacing with the charger, the ring 104 may activate one or more components of the PPG system 235. For example, the ring 104 may transmit, using an optical transmitter, light into a space between the charger and the ring 104 and receive, using an optical receiver, the transmitted light. The ring 104 may transmit the light for a specified duration of time. Upon receiving the light, the ring 104 may generate a signal representative of a signal level of the received light over the duration and using the processing module 230-a, determine a performance metric for the optical receiver/optical transmitter pair. The performance metric may include an average signal level of the signal or an average noise of the signal. The ring 104 may then send the performance metric to the user device 106 for further processing.

At the user device 106, the processing module 230-a may evaluate the performance metric. That is, the processing module 230-a may compare the performance metric to a baseline threshold. The baseline threshold may be an example of a baseline performance metric that is stored in memory of the user device 106 or at the server 110 and may include a previously determined performance metric or a performance metric that is based on optical measurement that were performed during manufacturing. The evaluation may indicate whether a change or update to the optical transmitter/optical receiver pair is needed. If a change is needed, the user device 106 may instruct the ring 104 to update one or more measurement parameters associated with the optical receiver/optical transmitter pair.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, which may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 where the optical receiver(s) receive transmitted light that is reflected through the region of the user's finger. In some implementations, the PPG system 235 may be configured as a transmissive PPG system 235 where the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBIs. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BMl160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In accordance with aspects described herein, the ring 104 may support one or more techniques for determining an optical performance of one or more components (e.g., components of the PPG system 235) and communicate a result of the determination and/or related signaling to the user device 106, which may display an indication of the optical performance and/or instructions for the user via a GUI 275.

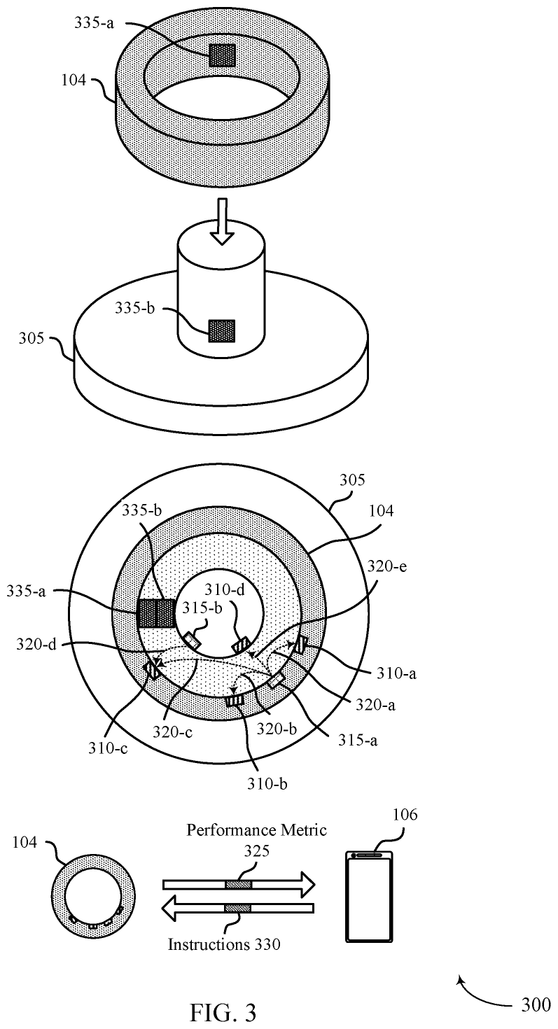

FIG. 3 shows an example of a system 300 that supports techniques for determining optical performance of a wearable device in accordance with aspects of the present disclosure. In some examples, the system 300 may implement, or be implemented by, aspects of a system 100 and a system 200. For example, the system 300 may include a wearable device 104 which may be an example of a wearable device 104 or a ring 104 as described with reference to FIGS. 1 and 2, respectively.

As shown in FIG. 3, the system 300 may include a wearable device 104 and a charging unit 305. FIG. 3 illustrates the wearable device 104 as a ring, however, it should be understood that the methods as described herein may be applied to any other type of wearable device 104 including, but not limited to, a smart watch. In some examples, the wearable device 104 may include an energy storage unit (e.g., a battery) that is operable to provide power to one or more electronic components of the wearable device 104. The energy storage unit may be coupled to an electrode 335-a that is positioned along an inner housing of the wearable device 104 (or an inner surface of a ring) and at least one surface of the electrode 335-a may be exposed (e.g., not covered by any material). As one example, the exposed surface of the electrode 335-a may be a surface of the electrode 335-a facing towards a center of the wearable device 104. In other examples, instead of, or in addition to the exposed electrodes 335, the wearable device 104 and the charging unit 305 may include inductance-based charging components or other components that transmit energy for charging without requiring direct contact.

During charging of the wearable device 104, the wearable device 104 may be placed on the charging unit 305. If the wearable device 104 is an example of a ring, the charging unit 305 may include a post. The post may be cylindrical in shape and an outside circumference of the post may be smaller than an inner circumference of the wearable device 104 such that, when the wearable device 104 is placed on the charging unit 305, the wearable device 104 may be concentric to the post of the charging unit 305. Further, the post of the charging unit 305 may include an electrode 335-b positioned on the outside surface (or a curved surface) of the post and at least one surface of the electrode 335-b may be exposed. As one example, the exposed surface of the electrode 335-b may be a surface of the electrode 335-b facing away from the center of the post. Further, the electrode 335-b may be coupled to (or wired to) an energy source (or a voltage source). When the wearable device 104 is placed on the charging unit 305, the exposed surface of the electrode 335-a may come in contact with the exposed surface of the electrode 335-b. Upon coupling the exposed surface of the electrode 335-a and the exposed surface of the electrode 335-b, charge may flow from the electrode 335-b to the electrode 335-a and further flow from the electrode 335-a to the energy storage unit.

If the wearable device 104 is an example of a wristable (e.g., a smart watch), the electrode 335-a may be situated on a surface of the wristable that is opposite from an outside surface of the wristable (e.g., a watch face of the smart watch or a surface that is opposite from the surface that is in contact with the user's wrist) and an exposed surface of the electrode 335-a may be a surface of the electrode 335-a that faces away from the outside surface. The charging unit 305 for the wristable may resemble a flat disk with the electrode 335-b situated on one of the flat surfaces of the disk. During charging, the charging unit 305 may be placed on the surface of the wristable that includes the electrode 335-a such that the electrode 335-a may interact with the electrode 335-b. In some examples, there may be a gap between the back of the watch face and the charging unit 305. In some examples, the wearable device 104 may be capable of wireless charging (e.g., optical charging). In such case, one or both of the wearable device 104 or the charging unit 305 may not include the electrodes 335.

As described herein, to validate the performance of optical components of the wearable device 104, one or both of the wearable device 104 or the charging unit 305 may perform optical measurements while charging. To perform the optical measurements, the wearable device 104 may include one or more optical components. For example, as shown in FIG. 3, the wearable device 104 may include a light-emitting component 315-a (e.g., an LED) as well as a photodetector 310-a, a photodetector 310-b, and a photodetector 310-c. Additionally or alternatively, the charging unit 305 may include one or more optical components. For example, as shown in FIG. 3, the charging unit 305 may include a light-emitting component 315-b as well as a photodetector 310-d. In some examples, the one or more light-emitting components 315 may be operable to transmit light corresponding to one or more wavelengths (e.g., IR light, red light, green light, or blue light) and the one or more photodetectors 310 may be operable to receive the light transmitted by the one or more light-emitting components 315.

While connected to the charging unit 305, the wearable device 104 may activate one or more of its optical components. For example, the wearable device 104 may activate the light-emitting component 315-a, the photodetector 310-a, the photodetector 310-b, and the photodetector 310-c, or any combination or sub-combination of these components. Upon activation, the light-emitting component 315-a may transmit light into a space between the wearable device 104 and the post of the charging unit 305 and the light may be received by the photodetector 310-a, the photodetector 310-b, and/or the photodetector 310-c. The light received by the different photodetectors 310 may travel along different optical paths 320. For example, the light received by the photodetector 310-a may travel along optical path 320-a, the light received by the photodetector 310-b may travel along optical path 320-b, and the light received by the photodetector 310-c may travel along optical path 320-c.

In some examples, the wearable device 104 may sequentially activate each of the photodetectors 310. For example, during the first duration, the wearable device 104 may activate the photodetector 310-a. During a second duration after the first duration, the wearable device 104 may activate the photodetector 310-b. During a third duration after the second duration, the wearable device 104 may activate the photodetector 310-c. In some examples, during all of the first duration, the second duration, and the third duration, the light-emitting component 315-a may be active and transmitting the light.

For each of the received light (the light received by the photodetector 310-a, the light received by the photodetector 310-b, and the light received by the photodetector 310-a), the wearable device 104 (or a processor of the wearable device 104) may generate a signal. In some examples, the signal may indicate a strength of the received light over time (e.g., a time that the respective photodetector 310 receives the light from the light-emitting component 315). Further, for each signal or each optical component pair, the wearable device 104 (or a processor of the wearable device 104) may determine a performance metric 325. The performance metric 325 may include one or both of an average of the signal strength of the respective signal or an average variation (or standard deviation) of the signal strength of the respective signal (e.g., otherwise known as noise). In some examples, the performance metrics 325 may collectively form a performance metric matrix. The performance matrix may represent the overall performance of the optical components of the wearable device 104.

Alternatively, while the wearable device 104 is connected to the charging unit 305, both the wearable device 104 and the charging unit 305 may activate one or more optical components. As one example, the wearable device 104 may activate the light-emitting component 315-a and the photodetector 310-c and the charging unit 305 may activate the light-emitting component 315-b and the photodetector 310-d. Upon activation, the light-emitting component 315-a may transmit first light into a space between the wearable device 104 and the post of the charging unit 305 and the first light may be received by the photodetector 310-d. Further, upon activation, the light-emitting component 315-b may transmit second light into a space between the wearable device 104 and the post of the charging unit 305 and the second light may be received by the photodetector 310-c. The second light received by the photodetector 310-c may travel along optical path 320-d and the first light received by the photodetector 310-d may travel along optical path 320-e. In some examples, the light-emitting component 315-a and the photodetector 310-d may be active during a different time interval than the light-emitting component 315-b and the photodetector 310-c.

The charging unit 305 may generate a first signal based on the first light received at the photodetector 310-d and the wearable device 104 may generate a second signal based on the second light received at the photodetector 310-c. The first signal and the second signal may indicate a strength of the respective received lights over time (e.g., a time that the respective photodetector 310 receives the light from the light-emitting component 315). Further, for the second signal, the wearable device 104 (or a processor of the wearable device 104) may determine a second performance metric 325 and for the first signal, the charging unit 305 may determine a first performance metric 325. In some examples, the charging unit 305 may transmit a signal to the wearable device 104 indicating the first performance metric 325 (e.g., using the light-emitting component 315-b). The first performance metric 325 and the second performance metric 325 may include one or both of an average of the signal strength of the respective signal or an average variation (or standard deviation) of the signal strength of the respective signal (e.g., otherwise known as noise).

In some examples, when performing the optical measurements or determining the performance metrics 325, the wearable device 104 may account for environmental factors that may negatively impact the optical measurements. These environmental factors may include ambient light, ambient temperature, motion of the wearable device 104, the spacing between the wearable device 104 and the post of the charging unit 305, a distance between the optical components (e.g., a ring size of the wearable device 104), or an orientation of the charging unit 305. As one example, the wearable device 104 may monitor motion data of the wearable device 104. If the motion data indicates that the wearable device 104 is moving, the wearable device 104 may dispose of the performance metrics 325 determined during the time of motion or the wearable device 104 may refrain from performing optical measurements for the purpose of determining the performance metrics 325 during the time of motion. In another example, if the optical measurements are performed during daytime, the wearable device 104 may apply an algorithm (e.g., an ambient light cancellation algorithm) to the optical measurement to account for the ambient light. In other examples, the wearable device 104 may perform the optical measurements during night such as to avoid the effects of the environmental factors.

Upon determining the performance metrics 325 for each optical component pair (e.g., light-emitting component 315 and photodiode 310 pair), the wearable device 104 may transmit a performance signal to a user device 106 associated with the user of the wearable device 104. The performance signal may indicate the performance metrics 325. In some examples, the wearable device 104 may periodically or aperiodically determine the performance metrics 325 and subsequently, send the performance metrics 325 to the user device 106. Upon receiving the performance signaling, the user device 106 (e.g., or an application of the user device 106) may measure the performance of the optical components of the wearable device 104 using the performance metrics. For example, the user device 106 may compare each of the performance metrics 325 to a respective threshold. In some examples, each of the respective thresholds may be based on a baseline performance metric 325 discovered during manufacturing (e.g., a performance metric 325 determined in the factory during testing of the wearable device 104). As one example, the user device 106 may compare a performance metric 325 corresponding to a first optical component pair (e.g., the light-emitting component 315-a and the photodetector 310-a) to a threshold that is based on a baseline performance metric 325 corresponding to the first optical component pair. In some examples, a threshold may be equal to a percentage of the respective baseline performance metric 325 (e.g., 75%, 85%, 95%, etc.). In some examples, the baseline performance metrics 325 or the thresholds may be stored in a memory of the user device 106.

If a performance metric 325 exceeds the respective threshold, the user device 106 may consider the performance of the optical component pair corresponding to the performance metric 325 satisfactory. Alternatively, if the performance metric 325 is below that respective threshold, the user device 106 may consider the performance of the optical component pair corresponding to the performance metric 325 unsatisfactory. After analyzing the performance of the optical components of the wearable device 104, the user device 106 may send instructions 330 to the wearable device 104 regarding the optical components of the wearable device 104. As one example, the user device 106 may instruct the wearable device 104 to refrain from using optical component pairs deemed as unsatisfactory to perform physiological measurements.

Alternatively, the user device 106 may instruct the wearable device 104 to update one or more measurement parameters associated with the optical components of an unsatisfactory optical component pair. For example, the user device 106 may instruct the wearable device 104 to update a power level (e.g., increase a transmit power) of a light-emitting component 315 while utilizing an unsatisfactory optical component pair including the light-emitting component 315, update a burn time (e.g., increase a burn time) of a light-emitting component 315 while utilizing an unsatisfactory optical component pair including the light-emitting component 315, or adjust an algorithm for analyzing light received at an photodetector 310 corresponding to an satisfactory optical component pair. In some examples, the wearable device 104 may apply the instructions 330 when performing measurements to obtain physiological data for the user of the wearable device 104. Applying such instructions 330 may allow the wearable device 104 to obtain more accurate physiological data for the user by taking into account the potential performance degradation of its optical components.

Additionally or alternatively, the user device 106 may track changes in the performance of the optical components of the wearable device 104. For example, at a first time, the user device 106 may receive a first performance metric 325 corresponding to an optical component pair from the wearable device 104 and at a second time, the user device 106 may receive a second performance metric 325 corresponding to the same optical component pair from the wearable device 104. In some examples, the user device 106 may store the first performance metric 325 and upon receiving the second performance metric, the user device 106 may compare the first performance metric 325 and the second performance metric 325 to determine a difference between the first performance metric 325 and the second performance metric. The user device 106 may than compare the difference (or an absolute value of the difference) to a threshold. If the difference is greater then the threshold, the user device 106 may identify that a performance of the optical component pair has changed over time. In some examples, upon detecting this change, the user device 106 may store a flag in its memory indicating the change or the user device 106 may transmit a signal to a server indicating that the performance of the optical component pair has changed.

In some examples, debris (e.g., dust or lint) may get onto the wearable device 104 or the charging unit 305. If the debris covers at least a portion of the optical component of the wearable device 104 or the charging unit 305, the performance of the optical components may be affected. For example, debris covering an light-emitting component 315 may block light being transmitted from the light-emitting component 315 resulting in less light being received at the one or more photodetectors 310. Thus, a change in the performance of an optical component of the wearable device 104 may be due to debris. In light of this, another action taken by the user device 106 may be to generate a display a message to the user instructing the user to clean one or both of the wearable device 104 or the charging unit 305 upon detecting unsatisfactory performance of an optical component pair or a change in the performance of an optical component pair.

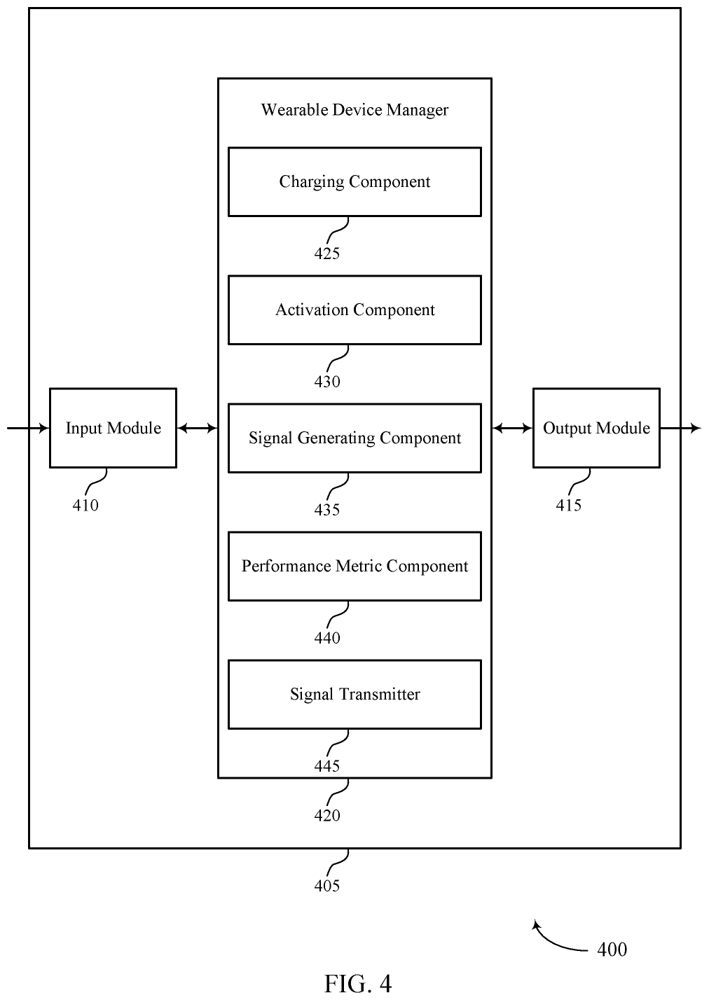

FIG. 4 shows a block diagram 400 of a device 405 that supports techniques for determining optical performance of a wearable device in accordance with aspects of the present disclosure. The device 405 may include an input module 410, an output module 415, and a wearable device manager 420. The device 405, or one or more components of the device 405 (e.g., the input module 410, the output module 415, and the wearable device manager 420), may include at least one processor, which may be coupled with at least one memory, to support the described techniques. Each of these components may be in communication with one another (e.g., via one or more buses).

For example, the wearable device manager 420 may include a charging component 425, an activation component 430, a signal generating component 435, a performance metric component 440, a signal transmitter 445, or any combination thereof. In some examples, the wearable device manager 420, or various components thereof, may be configured to perform various operations (e.g., receiving, monitoring, transmitting) using or otherwise in cooperation with the input module 410, the output module 415, or both. For example, the wearable device manager 420 may receive information from the input module 410, send information to the output module 415, or be integrated in combination with the input module 410, the output module 415, or both to receive information, transmit information, or perform various other operations as described herein.

The charging component 425 may be configured as or otherwise support a means for receiving, from a charging unit coupled with the wearable device, an electrical input for charging a battery of the wearable device. The activation component 430 may be configured as or otherwise support a means for activating, while the wearable device is coupled with the charging unit, one or more optical components of the wearable device. The signal generating component 435 may be configured as or otherwise support a means for generating a first signal based at least in part on light received at an optical component of the one or more optical components of the wearable device. The performance metric component 440 may be configured as or otherwise support a means for measuring a performance metric associated with the wearable device and based at least in part on the first signal, wherein the performance metric comprises one or both of an average of a signal strength of the first signal or an average variation of the signal strength of the first signal. The signal transmitter 445 may be configured as or otherwise support a means for transmitting a second signal indicating the performance metric to a user device associated with the wearable device.

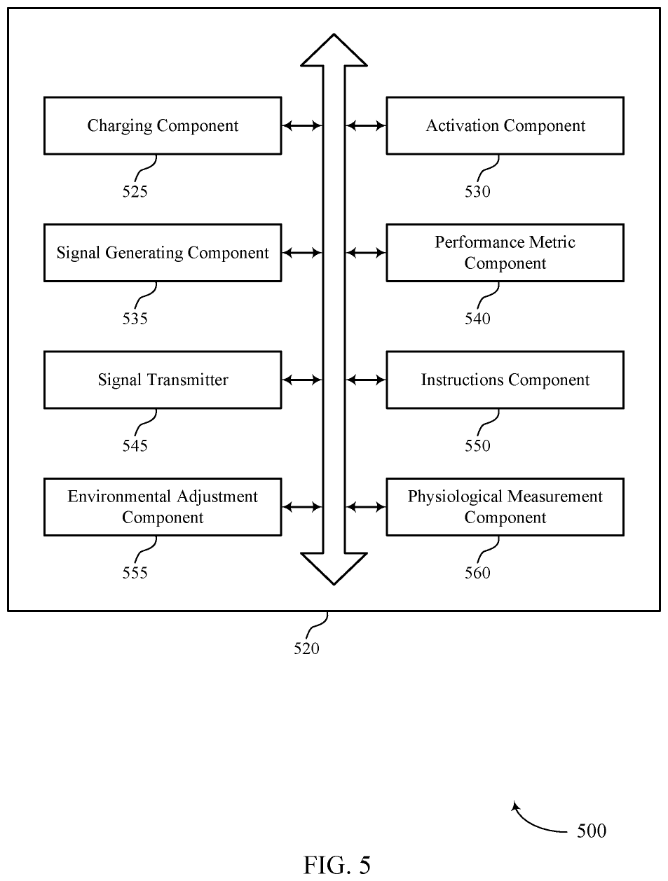

FIG. 5 shows a block diagram 500 of a wearable device manager 520 that supports techniques for determining optical performance of a wearable device in accordance with aspects of the present disclosure. The wearable device manager 520 may be an example of aspects of a wearable device manager or a wearable device manager 420, or both, as described herein. The wearable device manager 520, or various components thereof, may be an example of means for performing various aspects of techniques for determining optical performance of a wearable device as described herein. For example, the wearable device manager 520 may include a charging component 525, an activation component 530, a signal generating component 535, a performance metric component 540, a signal transmitter 545, an instructions component 550, an environmental adjustment component 555, a physiological measurement component 560, or any combination thereof. Each of these components, or components of subcomponents thereof (e.g., one or more processors, one or more memories), may communicate, directly or indirectly, with one another (e.g., via one or more buses).

The charging component 525 may be configured as or otherwise support a means for receiving, from a charging unit coupled with the wearable device, an electrical input for charging a battery of the wearable device. The activation component 530 may be configured as or otherwise support a means for activating, while the wearable device is coupled with the charging unit, one or more optical components of the wearable device. The signal generating component 535 may be configured as or otherwise support a means for generating a first signal based at least in part on light received at an optical component of the one or more optical components of the wearable device. The performance metric component 540 may be configured as or otherwise support a means for measuring a performance metric associated with the wearable device and based at least in part on the first signal, wherein the performance metric comprises one or both of an average of a signal strength of the first signal or an average variation of the signal strength of the first signal. The signal transmitter 545 may be configured as or otherwise support a means for transmitting a second signal indicating the performance metric to a user device associated with the wearable device.

In some examples, to support activating the one or more optical components, the activation component 530 may be configured as or otherwise support a means for transmitting, using a light-emitting component of the one or more light-emitting components of the wearable device, the light. In some examples, to support activating the one or more optical components, the activation component 530 may be configured as or otherwise support a means for receiving, using a first photodetector of the one or more photodetectors of the wearable device, the light, wherein generating the first signal is based at least in part on the light received at the first photodetector.

In some examples, to support activating the one or more optical components, the activation component 530 may be configured as or otherwise support a means for receiving, using a second photodetector of the one or more photodetectors of the wearable device, the light. In some examples, to support activating the one or more optical components, the signal generating component 535 may be configured as or otherwise support a means for generating a third signal based at least in part on the light received at the second photodetector. In some examples, to support activating the one or more optical components, the performance metric component 540 may be configured as or otherwise support a means for measuring a second performance metric associated with the wearable device based at least in part on the third signal, wherein the second performance metric comprises one or both of an average of a signal strength of the third signal or an average variation of the signal strength of the third signal.

In some examples, the charging unit comprises one or more light-emitting components and the one or more optical components comprises one or more photodetectors. In some examples, generating the first signal is based at least in part on the light transmitted from the one or more light-emitting components of the charging unit and received at the one or more photodetectors of the wearable device.

In some examples, the charging unit comprises one or more photodetectors and the one or more optical components comprises one or more light-emitting components, and the activation component 530 may be configured as or otherwise support a means for transmitting, using a light-emitting component of the one or more light-emitting components of the wearable device, second light. In some examples, the charging unit comprises one or more photodetectors and the one or more optical components comprises one or more light-emitting components, and the activation component 530 may be configured as or otherwise support a means for receiving, from the charging unit, a third signal indicating a second performance metric associated with the wearable device based at least in part on a second signal, the second performance metric comprising one or both of an average of a signal strength of the second signal or an average variation of the signal strength of the second signal, wherein the second signal is based on the second light received by a photodetector of the one or more photodetectors of the charging unit.

In some examples, the instructions component 550 may be configured as or otherwise support a means for receiving, based at least in part on transmitting the second signal, a third signal from the user device instructing the wearable device to adjust one or more measurement parameters associated with the wearable device.

In some examples, the one or more measurement parameters associated with the wearable device comprise an activation state of the one or more optical components, a power level associated with the one or more optical components, a burn time associated with the one or more optical components, an algorithm for analyzing the light received at the optical component, or a combination thereof.

In some examples, the physiological measurement component 560 may be configured as or otherwise support a means for acquiring physiological data associated with a user of the wearable device via the wearable device based at least in part on the third signal.

In some examples, the environmental adjustment component 555 may be configured as or otherwise support a means for determining one or more environmental parameters associated with the wearable device while the one or more optical components are active, the one or more environmental parameters including motion data associated with the wearable device, a size of the wearable device, a gap distance between the wearable device and the charging unit, an ambient light corresponding to the wearable device, or an ambient temperature corresponding to the wearable device, wherein measuring the performance metric associated with the wearable device is based at least in part on the determined one or more environmental parameters. In some examples, the wearable device comprises a wearable ring device.

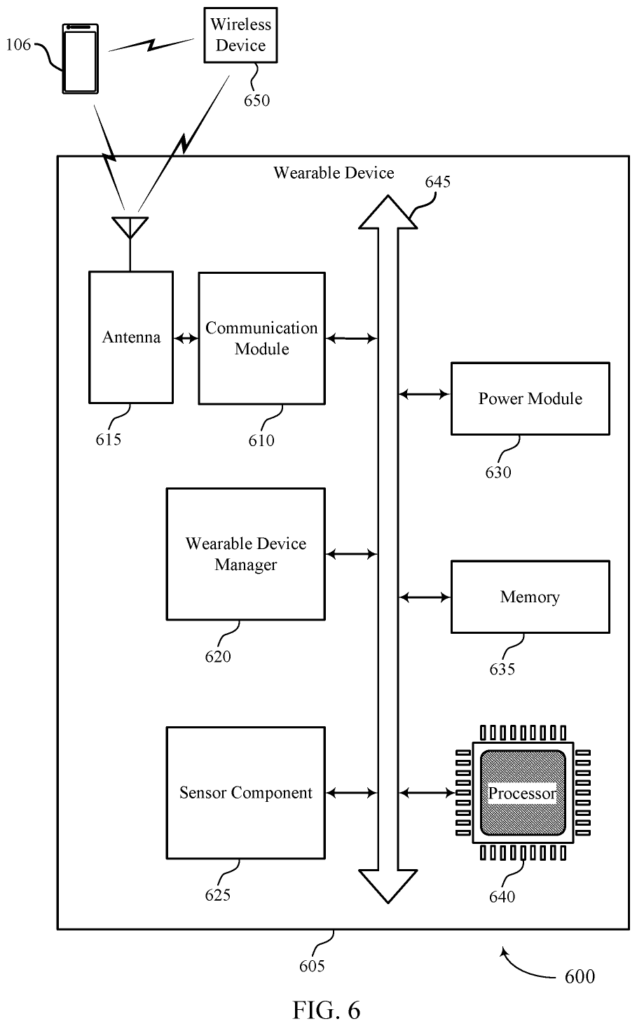

FIG. 6 shows a diagram of a system 600 including a device 605 that supports techniques for determining optical performance of a wearable device in accordance with aspects of the present disclosure. The device 605 may be an example of or include the components of a device 405 as described herein. The device 605 may include an example of a wearable device 104, as described previously herein. The device 605 may include components for bi-directional communications including components for transmitting and receiving communications with a user device 106 and a server 110, such as a wearable device manager 620, a communication module 610, an antenna 615, a sensor component 625, a power module 630, at least one memory 635, at least one processor 640, and a wireless device 650. These components may be in electronic communication or otherwise coupled (e.g., operatively, communicatively, functionally, electronically, electrically) via one or more buses (e.g., a bus 645).

For example, the wearable device manager 620 may be configured as or otherwise support a means for receiving, from a charging unit coupled with the wearable device, an electrical input for charging a battery of the wearable device. The wearable device manager 620 may be configured as or otherwise support a means for activating, while the wearable device is coupled with the charging unit, one or more optical components of the wearable device. The wearable device manager 620 may be configured as or otherwise support a means for generating a first signal based at least in part on light received at an optical component of the one or more optical components of the wearable device. The wearable device manager 620 may be configured as or otherwise support a means for measuring a performance metric associated with the wearable device and based at least in part on the first signal, wherein the performance metric comprises one or both of an average of a signal strength of the first signal or an average variation of the signal strength of the first signal. The wearable device manager 620 may be configured as or otherwise support a means for transmitting a second signal indicating the performance metric to a user device associated with the wearable device.

By including or configuring the wearable device manager 620 in accordance with examples as described herein, the device 605 may support techniques for increased accuracy of physiological data collected by the wearable device for the user.

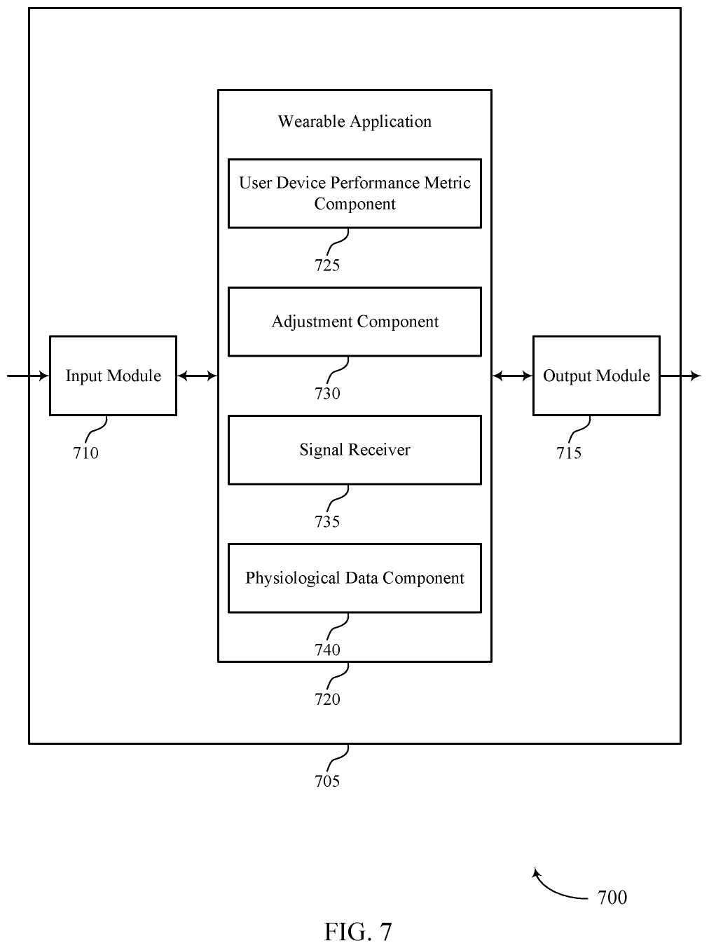

FIG. 7 shows a block diagram 700 of a device 705 that supports techniques for determining optical performance of a wearable device in accordance with aspects of the present disclosure. The device 705 may include an input module 710, an output module 715, and a wearable application 720. The device 705, or one or more components of the device 705 (e.g., the input module 710, the output module 715, and the wearable application 720), may include at least one processor, which may be coupled with at least one memory, to support the described techniques. Each of these components may be in communication with one another (e.g., via one or more buses).

The input module 710 may provide a means for receiving information such as packets, user data, control information, or any combination thereof associated with various information channels (e.g., control channels, data channels, information channels related to illness detection techniques). Information may be passed on to other components of the device 705. The input module 710 may utilize a single antenna or a set of multiple antennas.

The output module 715 may provide a means for transmitting signals generated by other components of the device 705. For example, the output module 715 may transmit information such as packets, user data, control information, or any combination thereof associated with various information channels (e.g., control channels, data channels, information channels related to illness detection techniques). In some examples, the output module 715 may be co-located with the input module 710 in a transceiver module. The output module 715 may utilize a single antenna or a set of multiple antennas.

For example, the wearable application 720 may include a user device performance metric component 725, an adjustment component 730, a signal receiver 735, a physiological data component 740, or any combination thereof. In some examples, the wearable application 720, or various components thereof, may be configured to perform various operations (e.g., receiving, monitoring, transmitting) using or otherwise in cooperation with the input module 710, the output module 715, or both. For example, the wearable application 720 may receive information from the input module 710, send information to the output module 715, or be integrated in combination with the input module 710, the output module 715, or both to receive information, transmit information, or perform various other operations as described herein.

The user device performance metric component 725 may be configured as or otherwise support a means for receiving, from a wearable device associated with the user device, a performance metric associated with the wearable device, the performance metric comprising one or both of an average of a signal strength of a first signal or an average variation of the signal strength of the first signal, wherein the first signal is generated by the wearable device using one or more optical components while the wearable device is coupled with a charging unit. The adjustment component 730 may be configured as or otherwise support a means for adjusting one or more measurement parameters associated with the wearable device based at least in part on a calibration process at the user device in response to receiving the performance metric. The signal receiver 735 may be configured as or otherwise support a means for receiving, from the wearable device, physiological data measured from a user by the wearable device. The physiological data component 740 may be configured as or otherwise support a means for computing one or more physiological parameters based at least in part on the adjusted one or more measurement parameters associated with the wearable device.

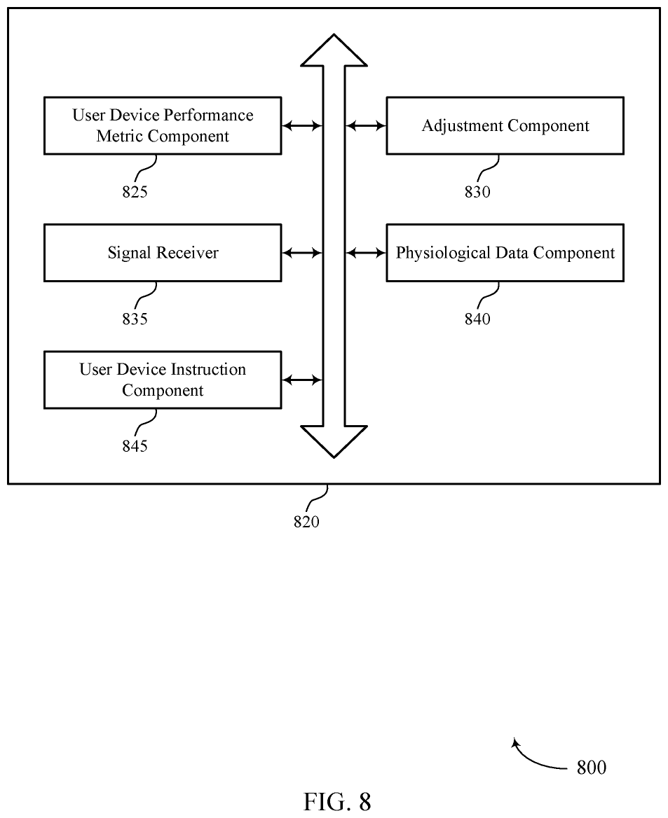

FIG. 8 shows a block diagram 800 of a wearable application 820 that supports techniques for determining optical performance of a wearable device in accordance with aspects of the present disclosure. The wearable application 820 may be an example of aspects of a wearable application or a wearable application 720, or both, as described herein. The wearable application 820, or various components thereof, may be an example of means for performing various aspects of techniques for determining optical performance of a wearable device as described herein. For example, the wearable application 820 may include a user device performance metric component 825, an adjustment component 830, a signal receiver 835, a physiological data component 840, a user device instruction component 845, or any combination thereof. Each of these components, or components of subcomponents thereof (e.g., one or more processors, one or more memories), may communicate, directly or indirectly, with one another (e.g., via one or more buses).

The user device performance metric component 825 may be configured as or otherwise support a means for receiving, from a wearable device associated with the user device, a performance metric associated with the wearable device, the performance metric comprising one or both of an average of a signal strength of a first signal or an average variation of the signal strength of the first signal, wherein the first signal is generated by the wearable device using one or more optical components while the wearable device is coupled with a charging unit. The adjustment component 830 may be configured as or otherwise support a means for adjusting one or more measurement parameters associated with the wearable device based at least in part on a calibration process at the user device in response to receiving the performance metric. The signal receiver 835 may be configured as or otherwise support a means for receiving, from the wearable device, physiological data measured from a user by the wearable device. The physiological data component 840 may be configured as or otherwise support a means for computing one or more physiological parameters based at least in part on the adjusted one or more measurement parameters associated with the wearable device.

In some examples, the user device performance metric component 825 may be configured as or otherwise support a means for comparing the performance metric to one or more thresholds, wherein adjusting the one or more measurement parameters is based at least in part on the performance metric satisfying at least one threshold of the one or more thresholds.

In some examples, the one or more thresholds are based at least in part on a second performance metric stored in a memory of the user device.

In some examples, the one or more measurement parameters comprise an activation state of the one or more optical components, a power level associated with the one or more optical components, a burn time associated with the one or more optical components, an algorithm for analyzing light received at the one or more optical components, or a combination thereof.

In some examples, the user device instruction component 845 may be configured as or otherwise support a means for transmitting a second signal instructing the wearable device to adjust the one or more measurement parameters.

In some examples, the user device instruction component 845 may be configured as or otherwise support a means for generating an instruction for the user to clean one or both of the wearable device of the charging unit based at least in part on the performance metric.

In some examples, the user device performance metric component 825 may be configured as or otherwise support a means for receiving, from the wearable device associated with the user device, a second performance metric associated with the wearable device, the second performance metric comprising one or both of an average of a signal strength of a second signal or an average variation of the signal strength of the second signal, wherein the second signal is generated by the wearable device using the one or more optical components while the wearable device is coupled with a charging unit, wherein adjusting the one or more measurement parameters is based at least in part on the second performance metric.

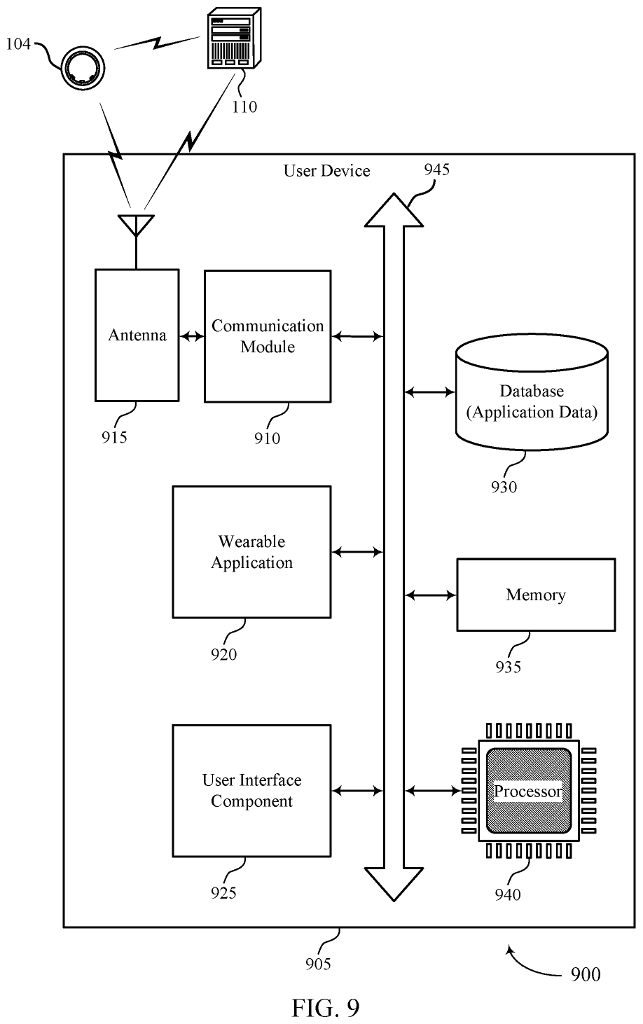

FIG. 9 shows a diagram of a system 900 including a device 905 that supports techniques for determining optical performance of a wearable device in accordance with aspects of the present disclosure. The device 905 may be an example of or include the components of a device 705 as described herein. The device 905 may include an example of a user device 106, as described previously herein. The device 905 may include components for bi-directional communications including components for transmitting and receiving communications with a wearable device 104 and a server 110, such as a wearable application 920, a communication module 910, an antenna 915, a user interface component 925, a database (application data) 930, at least one memory 935, and at least one processor 940. These components may be in electronic communication or otherwise coupled (e.g., operatively, communicatively, functionally, electronically, electrically) via one or more buses (e.g., a bus 945).

The communication module 910 may manage input and output signals for the device 905 via the antenna 915. The communication module 910 may include an example of the communication module 220-b of the user device 106 shown and described in FIG. 2. In this regard, the communication module 910 may manage communications with the ring 104 and the server 110, as illustrated in FIG. 2. The communication module 910 may also manage peripherals not integrated into the device 905. In some cases, the communication module 910 may represent a physical connection or port to an external peripheral. In some cases, the communication module 910 may utilize an operating system such as iOS®, ANDROID®, MS-DOS®, MS-WINDOWS®, OS/2®, UNIX®, LINUX®, or another known operating system. In other cases, the communication module 910 may represent or interact with a wearable device (e.g., ring 104), modem, a keyboard, a mouse, a touchscreen, or a similar device. In some cases, the communication module 910 may be implemented as part of the processor 940. In some examples, a user may interact with the device 905 via the communication module 910, user interface component 925, or via hardware components controlled by the communication module 910.

In some cases, the device 905 may include a single antenna 915. However, in some other cases, the device 905 may have more than one antenna 915, which may be capable of concurrently transmitting or receiving multiple wireless transmissions. The communication module 910 may communicate bi-directionally, via the one or more antennas 915, wired, or wireless links as described herein. For example, the communication module 910 may represent a wireless transceiver and may communicate bi-directionally with another wireless transceiver. The communication module 910 may also include a modem to modulate the packets, to provide the modulated packets to one or more antennas 915 for transmission, and to demodulate packets received from the one or more antennas 915.

The user interface component 925 may manage data storage and processing in a database 930. In some cases, a user may interact with the user interface component 925. In other cases, the user interface component 925 may operate automatically without user interaction. The database 930 may be an example of a single database, a distributed database, multiple distributed databases, a data store, a data lake, or an emergency backup database.

The memory 935 may include RAM and ROM. The memory 935 may store computer-readable, computer-executable software including instructions that, when executed, cause the processor 940 to perform various functions described herein. In some cases, the memory 935 may contain, among other things, a BIOS which may control basic hardware or software operation such as the interaction with peripheral components or devices.

The processor 940 may include an intelligent hardware device, (e.g., a general-purpose processor, a DSP, a CPU, a microcontroller, an ASIC, an FPGA, a programmable logic device, a discrete gate or transistor logic component, a discrete hardware component, or any combination thereof). In some cases, the processor 940 may be configured to operate a memory array using a memory controller. In other cases, a memory controller may be integrated into the processor 940. The processor 940 may be configured to execute computer-readable instructions stored in a memory 935 to perform various functions (e.g., functions or tasks supporting a method and system for sleep staging algorithms).

For example, the wearable application 920 may be configured as or otherwise support a means for receiving, from a wearable device associated with the user device, a performance metric associated with the wearable device, the performance metric comprising one or both of an average of a signal strength of a first signal or an average variation of the signal strength of the first signal, wherein the first signal is generated by the wearable device using one or more optical components while the wearable device is coupled with a charging unit. The wearable application 920 may be configured as or otherwise support a means for adjusting one or more measurement parameters associated with the wearable device based at least in part on a calibration process at the user device in response to receiving the performance metric. The wearable application 920 may be configured as or otherwise support a means for receiving, from the wearable device, physiological data measured from a user by the wearable device. The wearable application 920 may be configured as or otherwise support a means for computing one or more physiological parameters based at least in part on the adjusted one or more measurement parameters associated with the wearable device.

By including or configuring the wearable application 920 in accordance with examples as described herein, the device 905 may support techniques for increased accuracy of physiological data collected by the wearable device for the user.

The wearable application 920 may include an application (e.g., “app”), program, software, or other component which is configured to facilitate communications with a ring 104, server 110, other user devices 106, and the like. For example, the wearable application 920 may include an application executable on a user device 106 which is configured to receive data (e.g., physiological data) from a ring 104, perform processing operations on the received data, transmit and receive data with the servers 110, and cause presentation of data to a user 102.

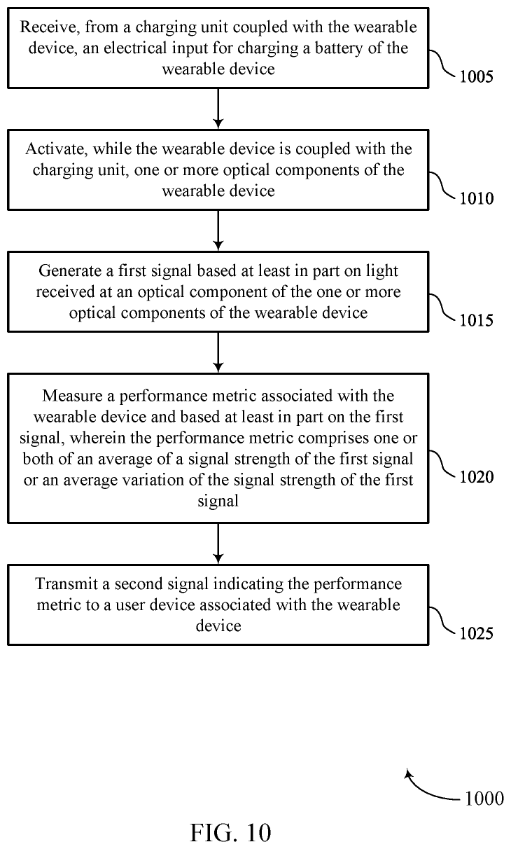

FIG. 10 shows a flowchart illustrating a method 1000 that supports techniques for determining optical performance of a wearable device in accordance with aspects of the present disclosure. The operations of the method 1000 may be implemented by a wearable device or its components as described herein. For example, the operations of the method 1000 may be performed by a wearable device as described with reference to FIGS. 1 through 6. In some examples, a wearable device may execute a set of instructions to control the functional elements of the wearable device to perform the described functions. Additionally, or alternatively, the wearable device may perform aspects of the described functions using special-purpose hardware.

At 1005, the method may include receiving, from a charging unit coupled with the wearable device, an electrical input for charging a battery of the wearable device. The operations of block 1005 may be performed in accordance with examples as disclosed herein. In some examples, aspects of the operations of 1005 may be performed by a charging component 525 as described with reference to FIG. 5.

At 1010, the method may include activating, while the wearable device is coupled with the charging unit, one or more optical components of the wearable device. The operations of block 1010 may be performed in accordance with examples as disclosed herein. In some examples, aspects of the operations of 1010 may be performed by an activation component 530 as described with reference to FIG. 5.

At 1015, the method may include generating a first signal based at least in part on light received at an optical component of the one or more optical components of the wearable device. The operations of block 1015 may be performed in accordance with examples as disclosed herein. In some examples, aspects of the operations of 1015 may be performed by a signal generating component 535 as described with reference to FIG. 5.

At 1020, the method may include measuring a performance metric associated with the wearable device and based at least in part on the first signal, wherein the performance metric comprises one or both of an average of a signal strength of the first signal or an average variation of the signal strength of the first signal. The operations of block 1020 may be performed in accordance with examples as disclosed herein. In some examples, aspects of the operations of 1020 may be performed by a performance metric component 540 as described with reference to FIG. 5.

At 1025, the method may include transmitting a second signal indicating the performance metric to a user device associated with the wearable device. The operations of block 1025 may be performed in accordance with examples as disclosed herein. In some examples, aspects of the operations of 1025 may be performed by a signal transmitter 545 as described with reference to FIG. 5.

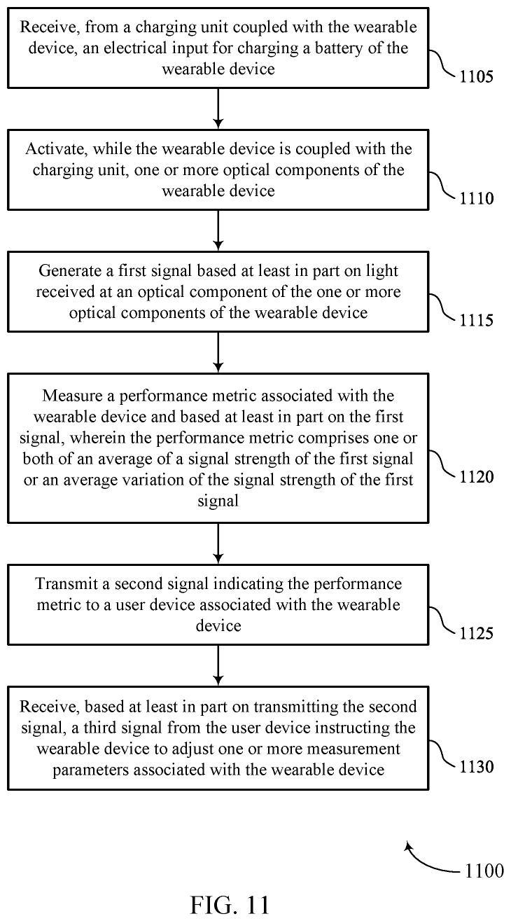

FIG. 11 shows a flowchart illustrating a method 1100 that supports techniques for determining optical performance of a wearable device in accordance with aspects of the present disclosure. The operations of the method 1100 may be implemented by a wearable device or its components as described herein. For example, the operations of the method 1100 may be performed by a wearable device as described with reference to FIGS. 1 through 6. In some examples, a wearable device may execute a set of instructions to control the functional elements of the wearable device to perform the described functions. Additionally, or alternatively, the wearable device may perform aspects of the described functions using special-purpose hardware.

At 1105, the method may include receiving, from a charging unit coupled with the wearable device, an electrical input for charging a battery of the wearable device. The operations of block 1105 may be performed in accordance with examples as disclosed herein. In some examples, aspects of the operations of 1105 may be performed by a charging component 525 as described with reference to FIG. 5.

At 1110, the method may include activating, while the wearable device is coupled with the charging unit, one or more optical components of the wearable device. The operations of block 1110 may be performed in accordance with examples as disclosed herein. In some examples, aspects of the operations of 1110 may be performed by an activation component 530 as described with reference to FIG. 5.

At 1115, the method may include generating a first signal based at least in part on light received at an optical component of the one or more optical components of the wearable device. The operations of block 1115 may be performed in accordance with examples as disclosed herein. In some examples, aspects of the operations of 1115 may be performed by a signal generating component 535 as described with reference to FIG. 5.

At 1120, the method may include measuring a performance metric associated with the wearable device and based at least in part on the first signal, wherein the performance metric comprises one or both of an average of a signal strength of the first signal or an average variation of the signal strength of the first signal. The operations of block 1120 may be performed in accordance with examples as disclosed herein. In some examples, aspects of the operations of 1120 may be performed by a performance metric component 540 as described with reference to FIG. 5.

At 1125, the method may include transmitting a second signal indicating the performance metric to a user device associated with the wearable device. The operations of block 1125 may be performed in accordance with examples as disclosed herein. In some examples, aspects of the operations of 1125 may be performed by a signal transmitter 545 as described with reference to FIG. 5.

At 1130, the method may include receiving, based at least in part on transmitting the second signal, a third signal from the user device instructing the wearable device to adjust one or more measurement parameters associated with the wearable device. The operations of block 1130 may be performed in accordance with examples as disclosed herein. In some examples, aspects of the operations of 1130 may be performed by an instructions component 550 as described with reference to FIG. 5.

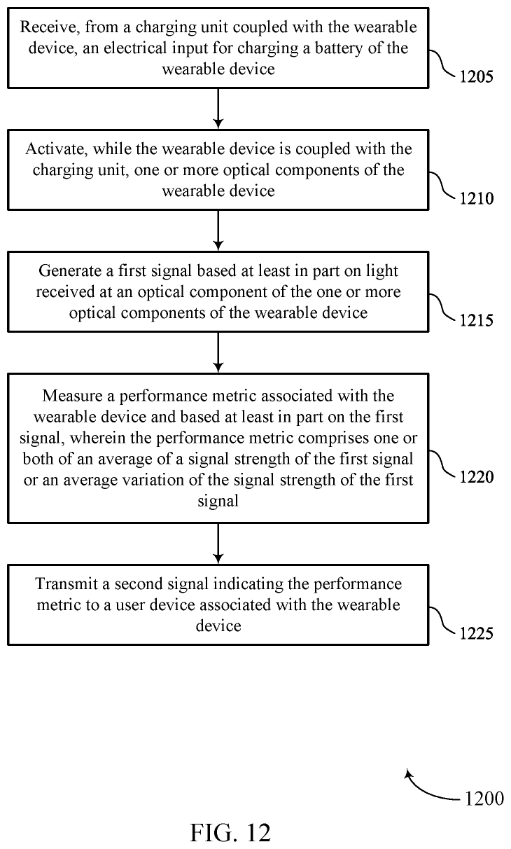

FIG. 12 shows a flowchart illustrating a method 1200 that supports techniques for determining optical performance of a wearable device in accordance with aspects of the present disclosure. The operations of the method 1200 may be implemented by a wearable device or its components as described herein. For example, the operations of the method 1200 may be performed by a wearable device as described with reference to FIGS. 1 through 6. In some examples, a wearable device may execute a set of instructions to control the functional elements of the wearable device to perform the described functions. Additionally, or alternatively, the wearable device may perform aspects of the described functions using special-purpose hardware.

At 1205, the method may include receiving, from a charging unit coupled with the wearable device, an electrical input for charging a battery of the wearable device. The operations of block 1205 may be performed in accordance with examples as disclosed herein. In some examples, aspects of the operations of 1205 may be performed by a charging component 525 as described with reference to FIG. 5.

At 1210, the method may include activating, while the wearable device is coupled with the charging unit, one or more optical components of the wearable device. The operations of block 1210 may be performed in accordance with examples as disclosed herein. In some examples, aspects of the operations of 1210 may be performed by an activation component 530 as described with reference to FIG. 5.

At 1215, the method may include generating a first signal based at least in part on light received at an optical component of the one or more optical components of the wearable device. The operations of block 1215 may be performed in accordance with examples as disclosed herein. In some examples, aspects of the operations of 1215 may be performed by a signal generating component 535 as described with reference to FIG. 5.

At 1220, the method may include measuring a performance metric associated with the wearable device and based at least in part on the first signal, wherein the performance metric comprises one or both of an average of a signal strength of the first signal or an average variation of the signal strength of the first signal. The operations of block 1220 may be performed in accordance with examples as disclosed herein. In some examples, aspects of the operations of 1220 may be performed by a performance metric component 540 as described with reference to FIG. 5.

At 1225, the method may include transmitting a second signal indicating the performance metric to a user device associated with the wearable device. The operations of block 1225 may be performed in accordance with examples as disclosed herein. In some examples, aspects of the operations of 1225 may be performed by a signal transmitter 545 as described with reference to FIG. 5.

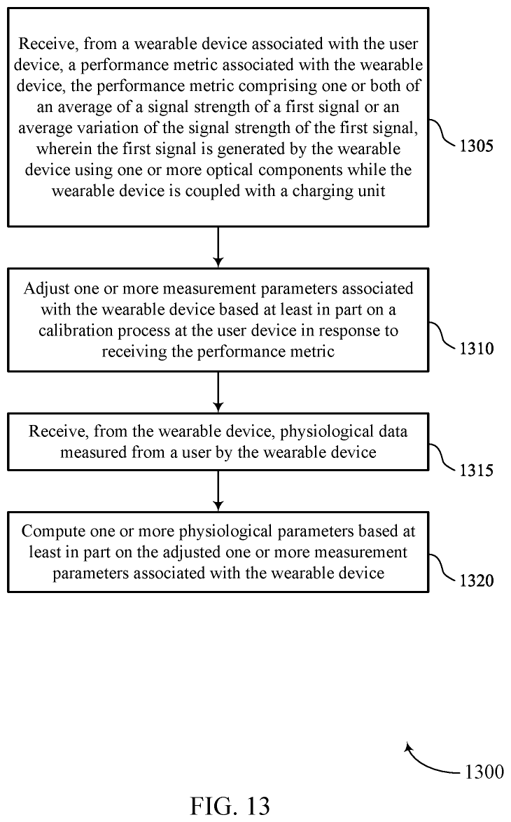

FIG. 13 shows a flowchart illustrating a method 1300 that supports techniques for determining optical performance of a wearable device in accordance with aspects of the present disclosure. The operations of the method 1300 may be implemented by a user device or its components as described herein. For example, the operations of the method 1300 may be performed by a user device as described with reference to FIGS. 1 through 3 and 7 through 9. In some examples, a user device may execute a set of instructions to control the functional elements of the user device to perform the described functions. Additionally, or alternatively, the user device may perform aspects of the described functions using special-purpose hardware.

At 1305, the method may include receiving, from a wearable device associated with the user device, a performance metric associated with the wearable device, the performance metric comprising one or both of an average of a signal strength of a first signal or an average variation of the signal strength of the first signal, wherein the first signal is generated by the wearable device using one or more optical components while the wearable device is coupled with a charging unit. The operations of block 1305 may be performed in accordance with examples as disclosed herein. In some examples, aspects of the operations of 1305 may be performed by a user device performance metric component 825 as described with reference to FIG. 8.

At 1310, the method may include adjusting one or more measurement parameters associated with the wearable device based at least in part on a calibration process at the user device in response to receiving the performance metric. The operations of block 1310 may be performed in accordance with examples as disclosed herein. In some examples, aspects of the operations of 1310 may be performed by an adjustment component 830 as described with reference to FIG. 8.

At 1315, the method may include receiving, from the wearable device, physiological data measured from a user by the wearable device. The operations of block 1315 may be performed in accordance with examples as disclosed herein. In some examples, aspects of the operations of 1315 may be performed by a signal receiver 835 as described with reference to FIG. 8.

At 1320, the method may include computing one or more physiological parameters based at least in part on the adjusted one or more measurement parameters associated with the wearable device. The operations of block 1320 may be performed in accordance with examples as disclosed herein. In some examples, aspects of the operations of 1320 may be performed by a physiological data component 840 as described with reference to FIG. 8.

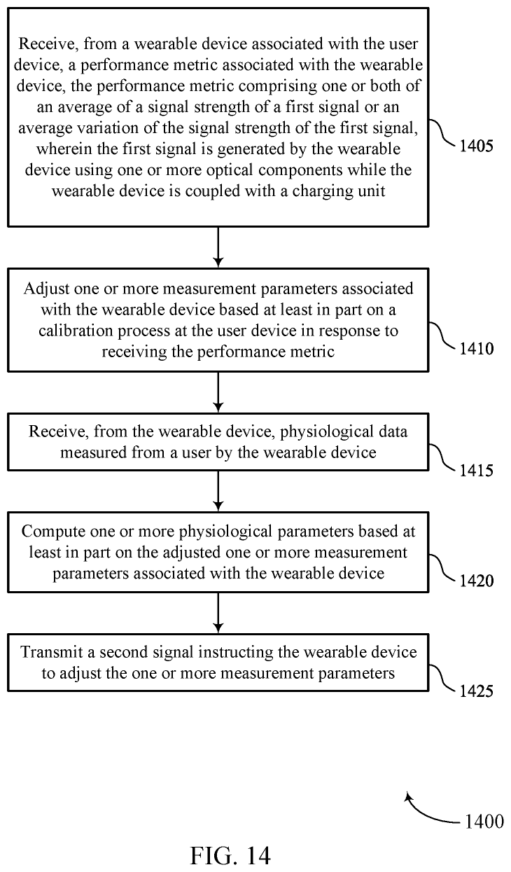

FIG. 14 shows a flowchart illustrating a method 1400 that supports techniques for determining optical performance of a wearable device in accordance with aspects of the present disclosure. The operations of the method 1400 may be implemented by a user device or its components as described herein. For example, the operations of the method 1400 may be performed by a user device as described with reference to FIGS. 1 through 3 and 7 through 9. In some examples, a user device may execute a set of instructions to control the functional elements of the user device to perform the described functions. Additionally, or alternatively, the user device may perform aspects of the described functions using special-purpose hardware.

At 1405, the method may include receiving, from a wearable device associated with the user device, a performance metric associated with the wearable device, the performance metric comprising one or both of an average of a signal strength of a first signal or an average variation of the signal strength of the first signal, wherein the first signal is generated by the wearable device using one or more optical components while the wearable device is coupled with a charging unit. The operations of block 1405 may be performed in accordance with examples as disclosed herein. In some examples, aspects of the operations of 1405 may be performed by a user device performance metric component 825 as described with reference to FIG. 8.

At 1410, the method may include adjusting one or more measurement parameters associated with the wearable device based at least in part on a calibration process at the user device in response to receiving the performance metric. The operations of block 1410 may be performed in accordance with examples as disclosed herein. In some examples, aspects of the operations of 1410 may be performed by an adjustment component 830 as described with reference to FIG. 8.

At 1415, the method may include receiving, from the wearable device, physiological data measured from a user by the wearable device. The operations of block 1415 may be performed in accordance with examples as disclosed herein. In some examples, aspects of the operations of 1415 may be performed by a signal receiver 835 as described with reference to FIG. 8.

At 1420, the method may include computing one or more physiological parameters based at least in part on the adjusted one or more measurement parameters associated with the wearable device. The operations of block 1420 may be performed in accordance with examples as disclosed herein. In some examples, aspects of the operations of 1420 may be performed by a physiological data component 840 as described with reference to FIG. 8.

At 1425, the method may include transmitting a second signal instructing the wearable device to adjust the one or more measurement parameters. The operations of block 1425 may be performed in accordance with examples as disclosed herein. In some examples, aspects of the operations of 1425 may be performed by a user device instruction component 845 as described with reference to FIG. 8.

It should be noted that the methods described above describe possible implementations, and that the operations and the steps may be rearranged or otherwise modified and that other implementations are possible. Furthermore, aspects from two or more of the methods may be combined.

A method by a wearable device is described. The method may include receiving, from a charging unit coupled with the wearable device, an electrical input for charging a battery of the wearable device, activating, while the wearable device is coupled with the charging unit, one or more optical components of the wearable device, generating a first signal based at least in part on light received at an optical component of the one or more optical components of the wearable device, measuring a performance metric associated with the wearable device and based at least in part on the first signal, wherein the performance metric comprises one or both of an average of a signal strength of the first signal or an average variation of the signal strength of the first signal, and transmitting a second signal indicating the performance metric to a user device associated with the wearable device.

A wearable device is described. The wearable device may include one or more memories storing processor executable code, and one or more processors coupled with the one or more memories. The one or more processors may individually or collectively operable to execute the code to cause the wearable device to receive, from a charging unit coupled with the wearable device, an electrical input for charging a battery of the wearable device, activate, while the wearable device is coupled with the charging unit, one or more optical components of the wearable device, generate a first signal based at least in part on light received at an optical component of the one or more optical components of the wearable device, measure a performance metric associated with the wearable device and based at least in part on the first signal, wherein the performance metric comprises one or both of an average of a signal strength of the first signal or an average variation of the signal strength of the first signal, and transmit a second signal indicating the performance metric to a user device associated with the wearable device.

Another wearable device is described. The wearable device may include means for receiving, from a charging unit coupled with the wearable device, an electrical input for charging a battery of the wearable device, means for activating, while the wearable device is coupled with the charging unit, one or more optical components of the wearable device, means for generating a first signal based at least in part on light received at an optical component of the one or more optical components of the wearable device, means for measuring a performance metric associated with the wearable device and based at least in part on the first signal, wherein the performance metric comprises one or both of an average of a signal strength of the first signal or an average variation of the signal strength of the first signal, and means for transmitting a second signal indicating the performance metric to a user device associated with the wearable device.

A non-transitory computer-readable medium storing code is described. The code may include instructions executable by a processor to receive, from a charging unit coupled with the wearable device, an electrical input for charging a battery of the wearable device, activate, while the wearable device is coupled with the charging unit, one or more optical components of the wearable device, generate a first signal based at least in part on light received at an optical component of the one or more optical components of the wearable device, measure a performance metric associated with the wearable device and based at least in part on the first signal, wherein the performance metric comprises one or both of an average of a signal strength of the first signal or an average variation of the signal strength of the first signal, and transmit a second signal indicating the performance metric to a user device associated with the wearable device.

In some examples of the method, wearable devices, and non-transitory computer-readable medium described herein, activating the one or more optical components may include operations, features, means, or instructions for transmitting, using a light-emitting component of the one or more light-emitting components of the wearable device, the light and receiving, using a first photodetector of the one or more photodetectors of the wearable device, the light, wherein generating the first signal may be based at least in part on the light received at the first photodetector.

In some examples of the method, wearable devices, and non-transitory computer-readable medium described herein, activating the one or more optical components may include operations, features, means, or instructions for receiving, using a second photodetector of the one or more photodetectors of the wearable device, the light, generating a third signal based at least in part on the light received at the second photodetector, and measuring a second performance metric associated with the wearable device based at least in part on the third signal, wherein the second performance metric comprises one or both of an average of a signal strength of the third signal or an average variation of the signal strength of the third signal.

In some examples of the method, wearable devices, and non-transitory computer-readable medium described herein, the charging unit comprises one or more light-emitting components and the one or more optical components comprises one or more photodetectors and generating the first signal may be based at least in part on the light transmitted from the one or more light-emitting components of the charging unit and received at the one or more photodetectors of the wearable device.

In some examples of the method, wearable devices, and non-transitory computer-readable medium described herein, the charging unit comprises one or more photodetectors and the one or more optical components comprises one or more light-emitting components and the method, apparatuses, and non-transitory computer-readable medium may include further operations, features, means, or instructions for transmitting, using a light-emitting component of the one or more light-emitting components of the wearable device, second light and receiving, from the charging unit, a third signal indicating a second performance metric associated with the wearable device based at least in part on a second signal, the second performance metric comprising one or both of an average of a signal strength of the second signal or an average variation of the signal strength of the second signal, wherein the second signal may be based on the second light received by a photodetector of the one or more photodetectors of the charging unit.

Some examples of the method, wearable devices, and non-transitory computer-readable medium described herein may further include operations, features, means, or instructions for receiving, based at least in part on transmitting the second signal, a third signal from the user device instructing the wearable device to adjust one or more measurement parameters associated with the wearable device.

In some examples of the method, wearable devices, and non-transitory computer-readable medium described herein, the one or more measurement parameters associated with the wearable device comprise an activation state of the one or more optical components, a power level associated with the one or more optical components, a burn time associated with the one or more optical components, an algorithm for analyzing the light received at the optical component, or a combination thereof.

Some examples of the method, wearable devices, and non-transitory computer-readable medium described herein may further include operations, features, means, or instructions for acquiring physiological data associated with a user of the wearable device via the wearable device based at least in part on the third signal.

Some examples of the method, wearable devices, and non-transitory computer-readable medium described herein may further include operations, features, means, or instructions for determining one or more environmental parameters associated with the wearable device while the one or more optical components may be active, the one or more environmental parameters including motion data associated with the wearable device, a size of the wearable device, a gap distance between the wearable device and the charging unit, an ambient light corresponding to the wearable device, or an ambient temperature corresponding to the wearable device, wherein measuring the performance metric associated with the wearable device may be based at least in part on the determined one or more environmental parameters.

In some examples of the method, wearable devices, and non-transitory computer-readable medium described herein, the wearable device comprises a wearable ring device.

A method by a user device is described. The method may include receiving, from a wearable device associated with the user device, a performance metric associated with the wearable device, the performance metric comprising one or both of an average of a signal strength of a first signal or an average variation of the signal strength of the first signal, wherein the first signal is generated by the wearable device using one or more optical components while the wearable device is coupled with a charging unit, adjusting one or more measurement parameters associated with the wearable device based at least in part on a calibration process at the user device in response to receiving the performance metric, receiving, from the wearable device, physiological data measured from a user by the wearable device, and computing one or more physiological parameters based at least in part on the adjusted one or more measurement parameters associated with the wearable device.

A user device is described. The user device may include one or more memories storing processor executable code, and one or more processors coupled with the one or more memories. The one or more processors may individually or collectively operable to execute the code to cause the user device to receive, from a wearable device associated with the user device, a performance metric associated with the wearable device, the performance metric comprising one or both of an average of a signal strength of a first signal or an average variation of the signal strength of the first signal, wherein the first signal is generated by the wearable device using one or more optical components while the wearable device is coupled with a charging unit, adjust one or more measurement parameters associated with the wearable device based at least in part on a calibration process at the user device in response to receiving the performance metric, receive, from the wearable device, physiological data measured from a user by the wearable device, and compute one or more physiological parameters based at least in part on the adjusted one or more measurement parameters associated with the wearable device.

Another user device is described. The user device may include means for receiving, from a wearable device associated with the user device, a performance metric associated with the wearable device, the performance metric comprising one or both of an average of a signal strength of a first signal or an average variation of the signal strength of the first signal, wherein the first signal is generated by the wearable device using one or more optical components while the wearable device is coupled with a charging unit, means for adjusting one or more measurement parameters associated with the wearable device based at least in part on a calibration process at the user device in response to receiving the performance metric, means for receiving, from the wearable device, physiological data measured from a user by the wearable device, and means for computing one or more physiological parameters based at least in part on the adjusted one or more measurement parameters associated with the wearable device.

A non-transitory computer-readable medium storing code is described. The code may include instructions executable by a processor to receive, from a wearable device associated with the user device, a performance metric associated with the wearable device, the performance metric comprising one or both of an average of a signal strength of a first signal or an average variation of the signal strength of the first signal, wherein the first signal is generated by the wearable device using one or more optical components while the wearable device is coupled with a charging unit, adjust one or more measurement parameters associated with the wearable device based at least in part on a calibration process at the user device in response to receiving the performance metric, receive, from the wearable device, physiological data measured from a user by the wearable device, and compute one or more physiological parameters based at least in part on the adjusted one or more measurement parameters associated with the wearable device.

Some examples of the method, user devices, and non-transitory computer-readable medium described herein may further include operations, features, means, or instructions for comparing the performance metric to one or more thresholds, wherein adjusting the one or more measurement parameters may be based at least in part on the performance metric satisfying at least one threshold of the one or more thresholds.

In some examples of the method, user devices, and non-transitory computer-readable medium described herein, the one or more thresholds may be based at least in part on a second performance metric stored in a memory of the user device.

In some examples of the method, user devices, and non-transitory computer-readable medium described herein, the one or more measurement parameters comprise an activation state of the one or more optical components, a power level associated with the one or more optical components, a burn time associated with the one or more optical components, an algorithm for analyzing light received at the one or more optical components, or a combination thereof.

Some examples of the method, user devices, and non-transitory computer-readable medium described herein may further include operations, features, means, or instructions for transmitting a second signal instructing the wearable device to adjust the one or more measurement parameters.

Some examples of the method, user devices, and non-transitory computer-readable medium described herein may further include operations, features, means, or instructions for generating an instruction for the user to clean one or both of the wearable device of the charging unit based at least in part on the performance metric.

Some examples of the method, user devices, and non-transitory computer-readable medium described herein may further include operations, features, means, or instructions for receiving, from the wearable device associated with the user device, a second performance metric associated with the wearable device, the second performance metric comprising one or both of an average of a signal strength of a second signal or an average variation of the signal strength of the second signal, wherein the second signal may be generated by the wearable device using the one or more optical components while the wearable device may be coupled with a charging unit, wherein adjusting the one or more measurement parameters may be based at least in part on the second performance metric.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A method of performing calibration at a wearable device, comprising:
receiving, from a charging unit coupled with the wearable device, an electrical input for charging a battery of the wearable device;
activating, while the wearable device is coupled with the charging unit, one or more optical components of the wearable device;
generating a first signal based at least in part on light received at an optical component of the one or more optical components of the wearable device;
measuring a performance metric associated with the wearable device based at least in part on the first signal, wherein the performance metric comprises one or both of an average of a signal strength of the first signal or an average variation of the signal strength of the first signal; and
transmitting a second signal indicating the performance metric to a user device associated with the wearable device.

2. The method of claim 1, wherein the one or more optical components comprise one or more light-emitting components and one or more photodetectors, and wherein activating the one or more optical components comprises:
transmitting, using a light-emitting component of the one or more light-emitting components of the wearable device, the light; and
receiving, using a first photodetector of the one or more photodetectors of the wearable device, the light, wherein generating the first signal is based at least in part on the light received at the first photodetector.

3. The method of claim 2, wherein activating the one or more optical components comprises:
receiving, using a second photodetector of the one or more photodetectors of the wearable device, the light;
generating a third signal based at least in part on the light received at the second photodetector; and
measuring a second performance metric associated with the wearable device based at least in part on the third signal, wherein the second performance metric comprises one or both of an average of a signal strength of the third signal or an average variation of the signal strength of the third signal.

4. The method of claim 1, wherein the charging unit comprises one or more light-emitting components and the one or more optical components comprises one or more photodetectors, and wherein generating the first signal is based at least in part on the light transmitted from the one or more light-emitting components of the charging unit and received at the one or more photodetectors of the wearable device.

5. The method of claim 1, wherein the charging unit comprises one or more photodetectors and the one or more optical components comprises one or more light-emitting components, the method further comprising:
transmitting, using a light-emitting component of the one or more light-emitting components of the wearable device, second light; and
receiving, from the charging unit, a third signal indicating a second performance metric associated with the wearable device based at least in part on a second signal, the second performance metric comprising one or both of an average of a signal strength of the second signal or an average variation of the signal strength of the second signal, wherein the second signal is based on the second light received by a photodetector of the one or more photodetectors of the charging unit.

6. The method of claim 1, further comprising:
receiving, based at least in part on transmitting the second signal, a third signal from the user device instructing the wearable device to adjust one or more measurement parameters associated with the wearable device.

7. The method of claim 6, wherein the one or more measurement parameters associated with the wearable device comprise an activation state of the one or more optical components, a power level associated with the one or more optical components, a burn time associated with the one or more optical components, an algorithm for analyzing the light received at the optical component, or a combination thereof.

8. The method of claim 6, further comprising:
acquiring physiological data associated with a user of the wearable device via the wearable device based at least in part on the third signal.

9. The method of claim 1, further comprising:
determining one or more environmental parameters associated with the wearable device while the one or more optical components are active, the one or more environmental parameters including motion data associated with the wearable device, a size of the wearable device, a gap distance between the wearable device and the charging unit, an ambient light corresponding to the wearable device, or an ambient temperature corresponding to the wearable device, wherein measuring the performance metric associated with the wearable device is based at least in part on the determined one or more environmental parameters.

10. The method of claim 1, wherein the wearable device comprises a wearable ring device.

11. A method of performing calibration at a user device, comprising:
receiving, from a wearable device associated with the user device, a performance metric associated with the wearable device, the performance metric comprising one or both of an average of a signal strength of a first signal or an average variation of the signal strength of the first signal, wherein the first signal is generated by the wearable device using one or more optical components while the wearable device is coupled with a charging unit;
adjusting one or more measurement parameters associated with the wearable device based at least in part on a calibration process at the user device in response to receiving the performance metric;
receiving, from the wearable device, physiological data measured from a user by the wearable device; and
computing one or more physiological parameters based at least in part on the adjusted one or more measurement parameters associated with the wearable device.

12. The method of claim 11, further comprising:
comparing the performance metric to one or more thresholds, wherein adjusting the one or more measurement parameters is based at least in part on the performance metric satisfying at least one threshold of the one or more thresholds.

13. The method of claim 12, wherein the one or more thresholds are based at least in part on a second performance metric stored in a memory of the user device.

14. The method of claim 11, wherein the one or more measurement parameters comprise an activation state of the one or more optical components, a power level associated with the one or more optical components, a burn time associated with the one or more optical components, an algorithm for analyzing light received at the one or more optical components, or a combination thereof.

15. The method of claim 11, further comprising:
transmitting a second signal instructing the wearable device to adjust the one or more measurement parameters.

16. The method of claim 11, further comprising:
generating an instruction for the user to clean one or both of the wearable device of the charging unit based at least in part on the performance metric.

17. The method of claim 11, further comprising:
receiving, from the wearable device associated with the user device, a second performance metric associated with the wearable device, the second performance metric comprising one or both of an average of a signal strength of a second signal or an average variation of the signal strength of the second signal, wherein the second signal is generated by the wearable device using the one or more optical components while the wearable device is coupled with a charging unit, wherein adjusting the one or more measurement parameters is based at least in part on the second performance metric.

18. A wearable device, comprising:
one or more memories storing processor-executable code; and
one or more processors coupled with the one or more memories and individually or collectively operable to execute the code to cause the wearable device to:
receive, from a charging unit coupled with the wearable device, an electrical input for charging a battery of the wearable device;
activate, while the wearable device is coupled with the charging unit, one or more optical components of the wearable device;
generate a first signal based at least in part on light received at an optical component of the one or more optical components of the wearable device;
measure a performance metric associated with the wearable device and based at least in part on the first signal, wherein the performance metric comprises one or both of an average of a signal strength of the first signal or an average variation of the signal strength of the first signal; and
transmit a second signal indicating the performance metric to a user device associated with the wearable device.

19. The wearable device of claim 18, wherein, to activate the one or more optical components, the one or more processors are individually or collectively operable to execute the code to cause the wearable device to:
transmit, using a light-emitting component of the one or more light-emitting components of the wearable device, the light; and
receive, using a first photodetector of the one or more photodetectors of the wearable device, the light, wherein generating the first signal is based at least in part on the light received at the first photodetector.

20. The wearable device of claim 18, wherein the charging unit comprises one or more light-emitting components and the one or more optical components comprises one or more photodetectors, and wherein generating the first signal is based at least in part on the light transmitted from the one or more light-emitting components of the charging unit and received at the one or more photodetectors of the wearable device.
